(19) United States
(12) Patent Application Publication
(10) Pub. No.: US 2021/0084851 A1
(43) Pub. Date: Mar. 25, 2021

(54) Generation of Dihaploids of Meadow Fescue and Festulolium

(71) Applicant: The United States of America, as Represented by the Secretary of Agriculture, Washington, DC (US)

(72) Inventor: Bryan K. Kindiger, El Reno, OK (US)

(21) Appl. No.: 16/575,792

(22) Filed: Sep. 19, 2019

(51) Int. Cl. A01H 1/08; A01H 5/10; A01H 6/46; A01H 1/02

(52) U.S. Cl. CPC A01H 1/08; A01H 1/02; A01H 6/4618; A01H 5/10

(57) ABSTRACT

Dihaploid recoveries of Schendonorus meadow fescue and festulolium may be created using the method disclosed herein. The method includes crossing a ryegrass (Lolium multiflorum) inducer line (IL) with individuals from a meadow fescue or festulolium cultivar or population. Such a cross can create a variety of resulting individual plants, including dihaploid recoveries. Some of the dihaploid recoveries can also be rhizomatous even when the parental meadow fescue or festulolium plant did not display a rhizomatous trait.

### BACKGROUND

Doubled haploids (dihaploids, DH) produced through in vivo induction of maternal haploids are completely homozygous and homogeneous for their genome constitution (Rotarenco, V. A. and S. T. Chalyk. 2000. Selection at the level of haploid sporophyte and its influence on the traits of diploid plants in maize. Genetika 32:479-485; Eder, J., and S. Chalyk. 2002. In vivo haploid induction in maize. Theor. Appl. Genet. 104:703-708; Rober, F. K., G. A. Gordillo, and H. H. Geiger. 2005. In vivo haploid induction in maize—Performance of new inducers and significance of doubled haploid lines in hybrid breeding. Maydica. 50:275-283; Chang, M. T., and E. H. Coe. 2009. Doubled haploids. pp. 127-142. In: A. L. Kriz and A. Larkins (eds). Biotechnology in Agriculture and Forestry. Vol. 63. Molecular Genetic Approaches to Maize Improvement. Springer Verlag, Berlin, Heidelberg; Geiger, H. H. 2009. Doubled haploids. pp. 641-659. In: J. L. Bennetzen and S. Hake (eds). Maize Handbook. Vol. II: Genetics and Genomics. Springer Verlag, Heidelberg, N.Y.). In addition, DH generation offers many enhancements and benefits for utilization across various breeding approaches (Snape, J. W., E. Simpson and B. B. Parker. 1986. Criteria for the selection and use of systems in cereal breeding programmes, pp: 217-229. In: W. Horn., C. J. Jensen., W. Odenbach. and O. Schieder (eds.), Genetic manipulation in plant breeding. Walter de Gruyter, Berlin; Maluszynski, M., K. J. Kasha, B. P. Forster and I. Szarejko. 2003. Doubled haploid production in crop plants: A manual. Kluwer Academic Publ., Dordrecht, Boston, London). DH production systems in breeding or selection programs represent a superior alternative to recurrent selection or mass selection approaches due to its increased efficiency during selection, reduction of the time for a breeding/selection cycle, the simplicity in their maintenance and their potential utilization in molecular marker-based trait mapping research (Martinez, V. A., W. G. Hill1 and S. A. Knott. 2002. On the use of double haploids for detecting QTL in outbred populations. Heredity 88:423-431; Tuvesson, S., S. C. Dayteg, P. Hagberg, O. Manninen, P. Tanhuanpaa”, T. Tenhola-Roininen, E. Kiviharju, J. Weyen, J. Forster, J. Schondelmaier, J. Lafferty, M. Marn, A. Fleck. 2007. Molecular markers and doubled haploids in European plant breeding programmes. Euphytica 58:305-312). In addition, the effectiveness of a DH selection approach is elevated when the number of genes governing a particular trait is quantitative in its inheritance and expression (Kotch, G. P.; R. Ortiz and S. J. Peloquin. 1992. Genetic analysis by use of potato haploid populations. Genome 35: 103-108). The promise in using a DH breeding approach resides in its capacity to facilitate the incorporation of desirable alleles within a shortened breeding period and the fact that no prior knowledge regarding the number of genes or inheritance of a trait is necessary (Singh, S. P. 1994. Gamete selection for simultaneous improvement of multiple traits in common bean. Crop Sci. 34:352-355; Bouchez, A. and A. Gallais. 2000. Efficiency of the use of doubled-haploids in recurrent selection for combining ability. Crop Sci. 40(1): 23-29). Established DH inducement systems exist for over 250 crop species (Forster, B. P., and W. T. B. Thomas. 2005. Doubled haploids in genetics and plant breeding. In: J. Janick (ed). Plant Breed Rev. 25:57-88); however, such an approach is lacking in most forage and turf grass species. The identification of a DH inducement approach would benefit the development of new forage or turf grass populations and/or cultivars.

Generally, haploid induction systems result in the generation of either maternal or paternal haploids when using a so called “inducer line.” It is this inducer line that provides the mechanism for the partial loss of a plant's genetic material or loss of entire genomes. In situations where maternal haploids represent the final product, the inducer line is utilized as the pollen parent (Fehr, W. 1984. Homozygous lines from double haploids. pp: 337-358. In: principles of cultivar development. Vol. 1. Macmillan Publishing Company, New York; Röber, et al., 2005). Conversely, when a paternal haploid or dihaploid is desireable, the inducer line is utilized as the female or seed parent (Genetic analysis of female gametophyte development and function. The Plant Cell Vol. 10 pp 5-17. 1998; Kermicle J. L. (1971). Pleiotropic effects on seed development of the indeterminate gametophyte gene in maize. Am. J. Bot. 58, 1-7).

Recently, tall fescue (TF, Schedonorus arundinaceus (Schreb.) Dumort., nom. cons.) DH lines have been generated utilizing an exceptional Lolium multiflorum inducer line (IL) (Kindiger, B. and D. Singh. 2011. Registration of Annual Ryegrass Genetic Stock IL2. J. of Plant Reg. 5:254-256; Kindiger, B. 2012. Notification of the Release of Annual Ryegrass Genetic Stock ILL J. of Plant Reg. 6:117-120; Kindiger, B. 2016. Generation of paternal dihaploids in tall fescue. Grassland Sci. 62:243-247; U.S. Pat. Nos. 8,618,353 and 8,912,388). These tall fescue DH are produced by the unique ability of the IL lines to induce genome instability in an IL/TF F1 hybrid. The genome instability results in the loss of the IL genome, leaving only the presence of a single dose of the TF genome in the apical meristem cell line. Spontaneous doubling occurs in that cell line providing for the chimera generation of a genetically homozygous, dihaploid (DH) tall fescue recovery. This behavior in the IL/TF hybridizations can either occur in the growing vegetative portions of the F1 plant or in the F1 plant inflorescence (Kindiger, 2016).

Meadow fescue (2n=2x=14) (Schendonorus pratensis (Huds.) P. Beauv.) is an outcrossing, self-incompatible species and represents a well utilized, introduced perennial cool-season forage grass in the USA. It exhibits wide adaptation, excellent spring, summer and fall production, a deep root system, and tolerance to heat. It responds well to fertilizer and has wide adaption across environments. These characteristics make this a highly desirable species for hay, pasture and turf. Utilizing the diploid (2n=2x=14) or, the induced tetraploid (4n=4x=28) forms, meadow fescue can be hybridized with both L. multiflorum and L. perenne to produce an array of novel breeding materials.

The development of superior meadow fescue cultivars has been limited to traditional recurrent or mass selection approaches, occasionally utilizing hybridization with L. multiflorum or L. perenne to introduce new genetic variation (Peto, F. H. 1933. The cytology of certain intergenetic hybrids between Lolium and Festuca. J. Genet. 28:113-157; Kleijer, G. 1984. Cytogenetic studies of crosses between Lolium multiflorum Lam. and Festuca arundinaceae, Schreb. I. The parents and their F1 hybrids. Z. Pflanzenzuchrg 93:1-22; Harper, J. Armstead, I., Thomas, A., James, C., Gasior, D., Bisaga, M., Roberts, L., King, I., and King, J. 2011. Alien introgression in the grasses Lolium perenne (perennial ryegrass) and Festuca pratensis (meadow fescue): the development of seven monosomic substitution lines and their molecular and cytological characterization. Ann. Bot. 8:1313-1321). Thus, it would be advantageous to develop a meadow fescue DH generation method utilizing a dihaploid inducer line.

All of the references cited herein, including U.S. Patents and U.S. Patent Application Publications, are incorporated by reference in their entirety.

Mention of trade names or commercial products in this publication is solely for the purpose of providing specific information and does not imply recommendation or endorsement by the U.S. Department of Agriculture.

### SUMMARY

The present invention relates to the creation of dihaploid recoveries of meadow fescue and/or festulolium. The method to create such recoveries involves the crossing of one or more meadow fescue plant(s) or festulolium plant(s) as the pollen parent with one or more Lolium multiflorum inducer line plant(s).

According to at least one embodiment of the invention, a method of producing dihaploid Schendonorus species plant material may include providing a L. multiflorum line which is capable of inducing genome loss (such as IL1 and IL2, described herein), crossing said L. multiflorum line as the maternal parent with a Schendonorus species utilized as the paternal parent to generate F1 interspecific hybrid plants, and identifying sectors (i.e. chimera sectors via genome loss of the inducer genome) in the F1 hybrid plants or in plants which are progeny thereof in which the sectors have a phenotype representative of Schendonorus comprising a dihaploid Schendonorus karyotype.

According to a further embodiment, a mature dihaploid Schendonorus plant can be grown from an F1 hybrid plant, progeny therefrom, or one of the above-described sectors.

According to a further embodiment, the Schendonorus species is meadow fescue, Schendonorus pratensis.

According to a further embodiment, any one of the F1 hybrid plants, progeny therefrom, or the mature dihaploid Schendonorus plant described above may contain or be capable of growing rhizomes.

According to another embodiment of the invention, dihaploid festulolium plant material may be produced by the same method as described above, except that sectors are identified in the F1 hybrid plants that have a phenotype representative of festulolium comprising a dihaploid festulolium karyotype. According to a further embodiment, a mature dihaploid festulolium plant may be grown from one of the F1 hybrid plants, progeny therefrom, or one of the recovered sectors.

Alternatively, dihaploid festulolium plant material may be produced by the same method as described above, except that the maternal parent is a festulolium plant, and said maternal parent is crossed with the L. multiflorum line which is capable of inducing genome loss.

According to a further embodiment, a dihaploid festulolium plant material may exhibit or be capable of growing rhizomes.

### BRIEF DESCRIPTION OF THE FIGURES

Advantages of embodiments of the present invention will be apparent from the following detailed description of the exemplary embodiments. The patent or application file contains at least one drawing executed in color. Copies of this patent or patent application publication with color drawing(s) will be provided by the office upon request and payment of the necessary fee.

The following detailed description should be considered in conjunction with the accompanying figures in which:

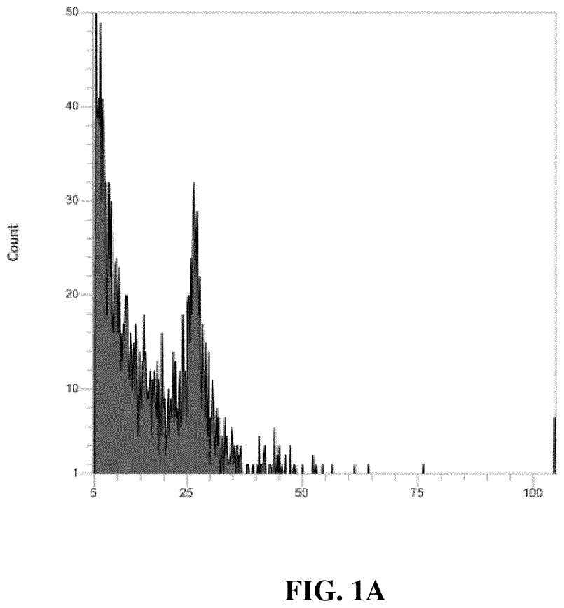

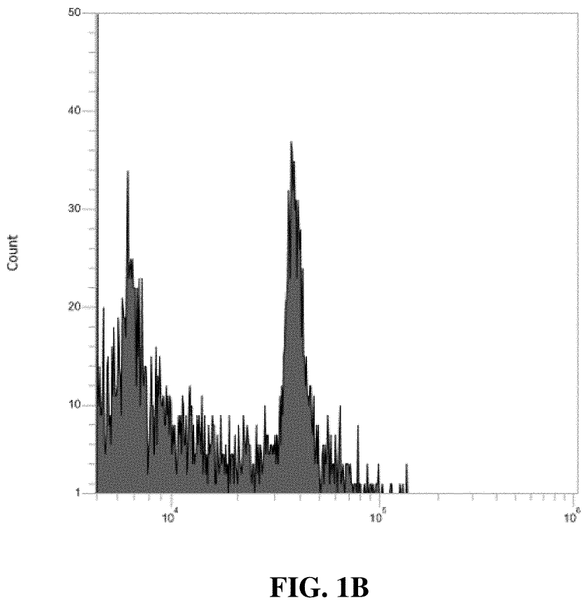

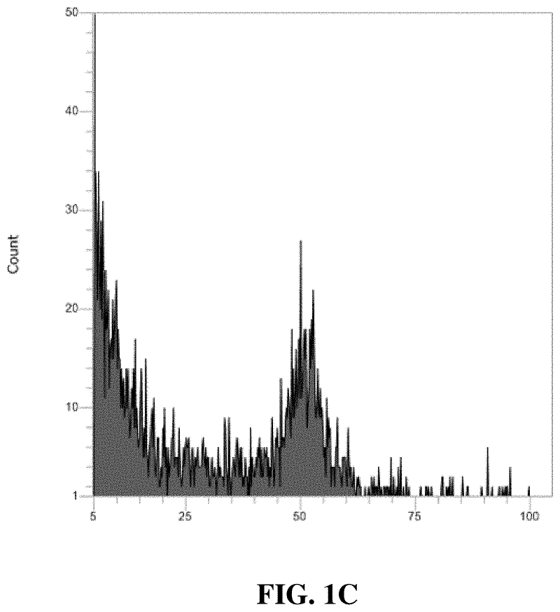

Exemplary FIG. 1A shows a flow cytometric image indicating multiple peaks (a chimera individual) exhibiting mitotic genome instability in the somatic leaf tissue of cell lines that are forming chimera sectors within individual leaves. The far-left peak is comparable to a haploid, 1n=7 ryegrass genome size. The peak at the right is comparable to an F1 hybrid between the ryegrass inducer and meadow fescue. The F1 peak has one diploid genomic dose of ryegrass and one of meadow fescue.

Exemplary FIG. 1B shows a flow cytometric image indicating multiple peaks (a chimera individual) indicating genome instability and the formation of cell lines or chimera sectors within individual leaves. The peak at the left is comparable to a cell line exhibiting a diploid, 2n=14 ryegrass genome size. The peak at the right is characteristic of nuclei exhibiting a genome size equivalent to meadow fescue dihaploid (MF DH).

Exemplary FIG. 1C shows a flow cytometric image indicating multiple peaks (chimera individual) indicating genome instability and the formation of cell lines or chimera sectors within individual leaves. The far-left peak estimates a cell line comparable to a haploid, 1n=14 ryegrass genome size. The peak at the right is characteristic of cell line nuclei exhibiting a genome size equivalent to meadow fescue dihaploid (MF DH).

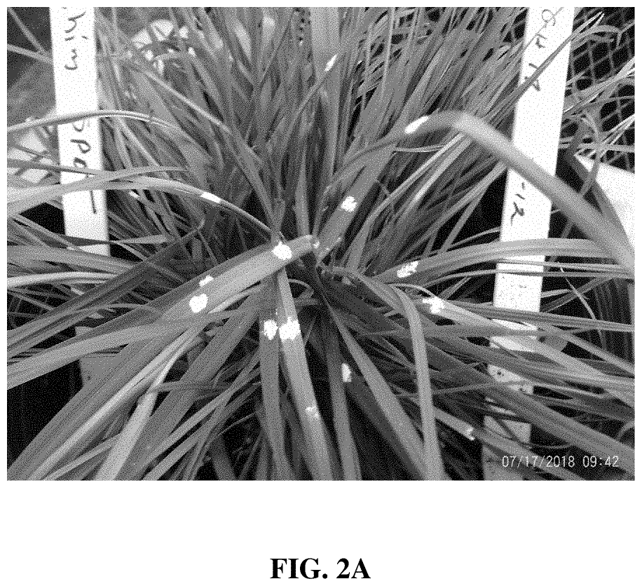

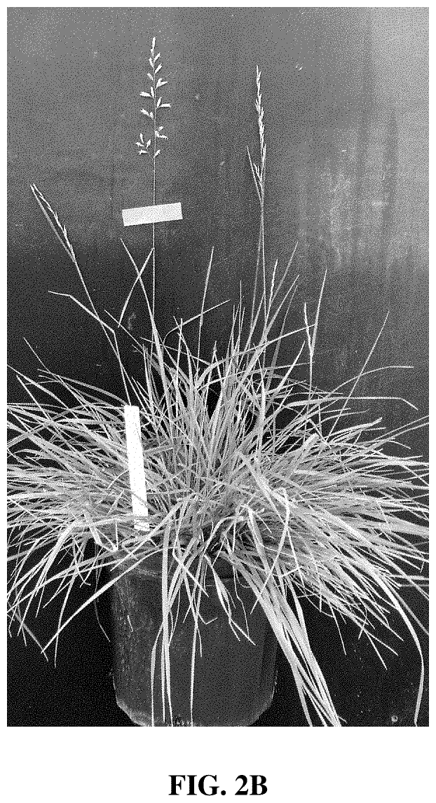

Exemplary FIG. 2A shows an image of an F1 hybrid exhibiting a meadow fescue type sector. Some leaves of the meadow fescue sector are identified with white spots to aid in the visual identification of the sector in the image.

Exemplary FIG. 2B shows an image of an example of a F1 hybrid exhibiting two different inflorescences. The inflorescence in the left-center is representative of a cell line sector representative of meadow fescue. The inflorescence in the right-center is representative of the inflorescence of the F1 region of the same plant.

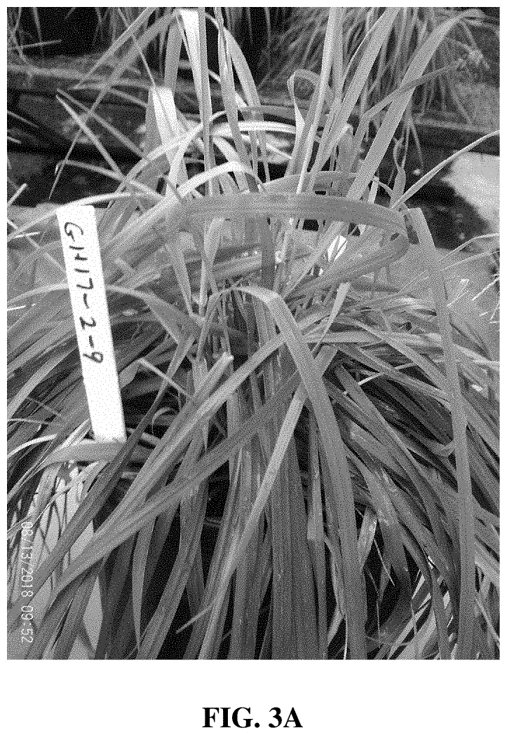

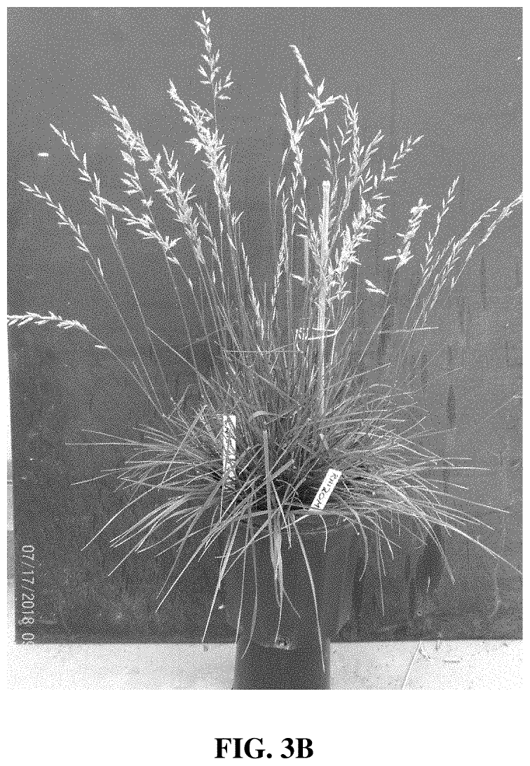

Exemplary FIG. 3A shows an image of a ryegrass-type phenotype that is not at all similar to the original IL ryegrass inducer maternal parent utilized in the hybridizations. Without being bound by theory, it is presumed that there was a random distribution of the ryegrass and meadow fescue chromosomes within the F1. The chimera sector that was formed following that distribution generated an individual distinctly different from ryegrass or meadow fescue (festulolium). The genomic constitution of this individual is presumed to be a mix of the IL ryegrass inducer and meadow fescue parental genomes.

Exemplary FIG. 3B shows an image of an unusual mature flowering plant exhibiting a distinct phenotype that is not similar to ryegrass, meadow fescue or the F1 hybrid. The genotype of this individual and flow cytometric data suggests this individual is a mix of the genomes of the IL ryegrass inducer and meadow fescue parents (festulolium).

### Statement Regarding Deposit of Biological Material Under the Terms of the Budapest Treaty

The inventors deposited samples of at least 2,500 seeds of each of the preferred ryegrass (L. multiflorum) inducer lines, IL1 and IL2, as described herein on or before Aug. 28, 2009, with the American Type Culture Collection (10801 University Blvd, Manassas, Va., 20110-2209, USA) in a manner affording permanence of the deposit and ready accessibility thereto by the public if a patent is granted. The deposit has been made under the terms of the Budapest Treaty on the International Recognition of the Deposit of Microorganisms for the Purposes of Patent Procedure and the regulations thereunder. The deposits' accession numbers are ATCC PTA-10229 (IL1) and ATCC PTA-10315 (IL2).

All restrictions on the availability to the public of L. multiflorum Accession Nos. ATCC PTA-10229 and ATCC PTA-10315 which have been deposited as described herein will be irrevocably removed upon the granting of a patent covering this particular biological material.

The L. multiflorum Accession Nos. ATCC PTA-10229 and ATCC PTA-10315 have been deposited under conditions such that access to the material is available during the pendency of the patent application to one determined by the Commissioner to be entitled thereto under 37 C.F.R. § 1.14 and 35 U.S.C. § 122.

The deposited biological material will be maintained with all the care necessary to keep them viable and uncontaminated for a period of at least five years after the most recent request for the furnishing of a sample of the deposited microorganisms, and in any case, for a period of at least thirty (30) years after the date of deposit or for the enforceable life of the patent, whichever period is longer.

We, the inventors for the invention described in this patent application, hereby declare further that all statements regarding this Deposit of the Biological Material made on information and belief are believed to be true and that all statements made on information and belief are believed to be true, and further that these statements are made with knowledge that willful false statements and the like so made are punishable by fine or imprisonment, or both, under section 1001 of Title 18 of the United States Code and that such willful false statements may jeopardize the validity of the instant patent application or any patent issuing thereon.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention,” “embodiments,” or “invention” do not require that all embodiments of the invention include the discussed feature, advantage or mode of operation.

Unless defined otherwise, all technical and scientific terms used herein have the same meaning as commonly understood by one of ordinary skill in the art to which the invention belongs. As used herein, the term “about” refers to a quantity, level, value, or amount that varies by as much as 30%, preferably by as much as 20%, and more preferably by as much as 10% to a reference quantity, level, value, or amount. Although any methods and materials similar or equivalent to those described herein can be used in the practice or testing of the present invention, the preferred methods and materials are now described.

The amounts, percentages, and ranges disclosed herein are not meant to be limiting, and increments between the recited amounts, percentages, and ranges are specifically envisioned as part of the invention.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein.

The present invention relates to the creation of dihaploid (DH) meadow fescue (MF), DH festulolium, and non-DH festulolium using one or more Lolium multiflorum inducer line(s). The invention is further illustrated by the following non-limiting Examples.

### Examples

### Methods and Materials

In 2002, an L. multiflorum (2n=2x=14) population was observed to segregate various levels of pollen sterility. Prior research suggested that selections from within this population were capable of inducing somatic genome loss through mitotic divisions. Further selection produced two inducer lines capable of inducing genome instability in intra- and inter-specific hybrids and these were given the designations IL1 and IL2. IL1 and IL2 were released in 2011 and 2012 (Kindiger and Singh, 2011; Kindiger, 2012).

In 2017, three IL lines (IL1, IL2, and IL3, of which IL1 and IL2 are the preferred lines) were allowed to be bulk pollinated by a meadow fescue cultivar (experimental designation FpF79) in an experimental seed production nursery at the Barenbrug Seeds, West Coast Research Laboratory, Albany, Oreg. USA. IL×FpF79 hybrids were produced by placing the IL lines randomly within the FpF79 seed production nursery. At IL maturity, seed from the IL lines were harvested and transferred to the USDA-ARS, Grazinglands Research Laboratory, El Reno, Okla. USA. Methods earlier utilized to generate tall fescue DH recoveries described elsewhere (Kindiger, 2016) were also used here. Briefly, seed were cleaned by hand and sown to germination trays in the greenhouse in October, 2017. By December, phenotypic differences among the seedlings were observable. Seedlings not exhibiting the typical IL inducer phenotype were removed and transferred to three inch pots. This class of seedlings are generally considered to represent F1 hybrid IL/FpF79 seedlings or early DH recoveries. Seedlings exhibiting the typical IL phenotype were deemed to be likely rarely occurring selfs or IL DH recoveries and were set aside. As the transplanted seedlings matured, seedlings were transferred to larger pots. The IL×FpF79 hybrids were allowed to grow to maturity, with weekly examinations for potential chimera sectors or other indicators of genome loss. Leaf samples from the retained seedlings were submitted to flow cytometry evaluation to confirm the incidence of genome instability and chimera sectoring.

### Plant Material Preparation and Flow Cytometry

In 2018, mature leaf samples from the greenhouse grown materials described above were obtained from each F1 individual to determine if the degree of any potential genome or somatic loss could be detected. In many instances, multiple leaf samples were obtained from visually obvious plant sectors.

For the flow cytometric analysis, approximately 0.05 g of fresh cut leaf tissue were placed in 1.5 ml Eppendorf tubes. Approximately 0.05 g of a 0.9-2.0 maceration stainless steel bead product (SSB14B, Next Advance Inc., Averill Park, N.Y., USA) and one 3.2 mm stainless steel bead (SSB32, Next Advance Inc., Averill Park, N.Y., USA) were combined for leaf maceration. 500 ul of Galbraith solution was placed in each tube (Galbraith D W, Harkins K R, Maddox J M, Ayres N M, Sharma D P, Firoozabady E. (1983) Rapid flow cytometric analysis of the cell-cycle in intact plant-tissues. Science 220: 1049-1051) and each sample was placed in a rotary bullet blender tissue homogenizer (Next Advance Inc., Averill Park, N.Y., USA) to macerate the leaf tissue.

Approximately 400 ul of this fluid were transferred from the Eppendorf tubes to 15 ml Corning tubes. Nuclei labelling and detection was achieved by dispensing 1 ml of FxCycle PI/RNase staining solution (Invitrogen by Thermo Fisher Scientific, 81 Wyman Street, Waltham, Mass. 02451 USA) into the macerated leaf tissue for one hour. Following the manufacturer's staining recommendations, samples were retained in darkness during the one-hour staining interval. Prior to flow cytometric analysis each sample was filtered through a 50 um CellTrics disposable filter (Sysmex-Partec GmbH, Goerlitz, Germany) before evaluations in a Life Technologies Attune NxT Acoustic Focusing Flow Cytometer (Model AFC2, Thermofisher Scientific, 81 Wyman Street, Waltham, Mass. 02451 USA). To provide for a base line estimator for various 2n=2x=14 genome size estimations, the IL1 inducer line and meadow fescue cultivars FpF79 and Pradel were used as checks.

### Dihaploid Formation Results

From the original IL×FpF79 hybridizations, 55 seedlings were selected and evaluated. Each retained individual was classified by its phenotype to exhibit an IL inducer ryegrass phenotype, an F1 phenotype, a diminutive phenotype, a festulolium phenotype or a meadow fescue phenotype (Table 1). It is noted that though a hybrid plant may itself be designated an “F1 plant,” the characterizations in the following table relate to the phenotypical characterizations, where an F1 plant may have one of several phenotypes, including the characteristic F1 phenotype.

TABLE 1
Phenotypes created from IL-MF hybridization

| | Dihaploid | # Individual | Rhizomes |
|---|---|---|---|
| Phenotype | (DH)? | plants | observed? |
| F1 | no | 9 | none |
| F1/MF Chimera | yes | 4 | none |
| MF | yes | 14 | 9 |
| IL | yes | 20 | none |
| Festulolium | yes | 6 | 1 |
| Diminutive | not tested | 2 | not tested |
| Total | | 55 | |

From this set of individuals, twenty were identified as IL selfs or IL DH recoveries. No additional evaluations were performed on these materials. Fourteen individuals exhibited a meadow fescue (MF) phenotype. These individuals likely arose from very early chimera formation or somatic genome loss in the initial apical meristem of the developing seedling. That is, these individuals had a very early loss of the IL genome. These individuals never exhibited an F1 phenotype and from seedlings grew into individuals exhibiting a meadow fescue phenotype. Thirteen individuals expressed the F1 phenotype. F1 IL×FpF79 hybrids were vigorous plants having very narrow, dark blue leaves. Inflorescences were intermediate between ryegrass and meadow fescue types. Flow cytometric evaluations indicated a strong level of genome instability in all these materials (e.g. as shown in FIGS. 1A-1C). Of these, four developed late-developing chimera meadow fescue sectors. Three individuals exhibited a ryegrass phenotype, and were strikingly different than the original IL parental phenotype. An additional three individuals exhibited an intermediate phenotype, strikingly different than meadow fescue or the F1. These two classes of individuals strongly suggest a mixoploid genome constitution with chromosomes or genetic contributions from both the IL and FpF79 parents. Likely, these individuals represent six DH festulolium recoveries (FIGS. 3A and 3B). Two, very slow, diminutive appearing plants were dissimilar to all the phenotypes discussed above. Due to their poor vitality and slow development, they had no commercial value and further evaluations were terminated on these individuals.

In addition to the above observations, as shown in Table 1, several of the individual seedlings were observed to generate rhizomes, a trait not found in the parent MF.

Flow cytometry evaluations were performed on all the individuals exhibiting a ryegrass/festulolium, F1, F1/MF-chimeral, or meadow fescue genotype. The flow cytometry analysis performed on the various individuals allowed the correlation of phenotype to a particular genome size estimation. These data were utilized to place the individuals into the ryegrass, F1, meadow fescue, or festulolium phenotypic categories. Most of these individuals exhibited repeatable and distinct flow cytometry profiles suggesting genome loss or randomness across genome constitutions was not likely. That is, the phenotypes described above are predictable and repeatable.

In some instances, visual examination of the F1 individuals during the vegetative growth stages revealed defined chimeral sectors. In one instance, a chimera sector exhibited a meadow fescue inflorescence that was adjacent to a typical F1 inflorescence (FIG. 2B).

Based on the present work, and prior work discussed above relating to the creating of DH TF, the most appropriate method for producing DH meadow fescue or DH festulolium lines appears to be restricted to discovering and isolating the chimeral sectors from an original F1 plant. As the degree of chimera sector induction appears high in these IL×FpF79 F1 hybrids, the level of DH generation via selection of the chimera meadow fescue type sectors represents an efficient and leisure strategy for DH generation. Results from Table 1 clearly indicate that 18 DH with a meadow fescue phenotype and exhibiting a meadow fescue genome size via flow cytometry occur at a level useful for commercial application.

In total, eighteen DH meadow fescue type recoveries were obtained during the study. Since each DH is a product of a single FpF79 pollen grain/sperm nuclei during fertilization of the IL line, each DH MF represents a unique genotype. Fourteen DH MF were derived through very early genome loss following the germination stage and four were identified as MF chimera sectors in the F1 individuals.

### Rhizomatous Meadow Fescue

It is noted, as stated above, that the transfer of the Lolium cytoplasm via the dihaploid generation process can result in the recovery of Schendonorus or festulolium dihaploids expressing or capable of expressing rhizomes. Thus, because the parent Schendonorus is incapable of growing rhizomes, a recovery which is capable of growing rhizomes would contain the Lolium cytoplasm from the maternal parent.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. A method of producing dihaploid Schedonorus species plant material, comprising:
(a) providing a Lolium multiflorum line which is capable of inducing genome loss;
(b) crossing said L. multiflorum line as the maternal parent with a Schedonorus species utilized as the paternal parent to generate F1 interspecific hybrid plants;
(c) identifying sectors in said F1 hybrid plants or in plants which are progeny thereof in which said sectors have a phenotype representative of Schedonorus comprising a dihaploid Schedonorus karyotype; and
(d) recovering said sectors identified in (c),
wherein said sectors identified in (c) are capable of developing into mature Schedonorus plants, and
wherein said Schedonorus species utilized as the paternal parent is not capable of growing rhizomes and at least one of said F1 hybrid plants is capable of growing rhizomes.

2. The method of claim 1, wherein the L. multiflorum line is selected from the group consisting of IL1 (ATCC deposit accession no. PTA-10229) and progeny thereof, and IL2 (ATCC deposit accession no. PTA-10315) and progeny thereof.

3. The method of claim 1, further comprising growing a mature dihaploid Schedonorus plant from at least one of: one of said F1 hybrid plants or progeny therefrom, or one of said sectors recovered in (d).

4. The method of claim 1, wherein said Schedonorus species is meadow fescue, Schedonorus pratensis.

5. The method of claim 1, wherein at least one of said F1 hybrid plants comprises cytoplasm of L. multiflorum.

6. (canceled)

7. A dihaploid Schedonorus species plant material produced by the method of claim 1.

8. A method of producing a dihaploid F1 hybrid plant, comprising:
(a) providing a Lolium multiflorum line which is capable of inducing genome loss;
(b) crossing said L. multiflorum line as the maternal parent with a Schedonorus species utilized as the paternal parent to generate interspecific hybrid plants; and
(c) recovering at least one of said F1 hybrid plants that comprises at least one sector having a phenotype representative of Schedonorus comprising a dihaploid Schedonorus karyotype,
wherein said Schedonorus species utilized as the paternal parent is not capable of growing rhizomes and at least one of said F1 hybrid plants is capable of growing rhizomes.

9. The method of producing a dihaploid Schendonorus species plant of claim 8, further comprising:
(d) providing the diploid dihaploid F1 plant of step (c);
(e) growing a mature dihaploid Schedonorus plant from at least one of: the F1 hybrid plant, progeny therefrom, or the at least one of said sectors having a phenotype representative of Schedonorus comprising a dihaploid Schedonorus karyotype.

10. The method of claim 8, wherein said Schendonorus Schedonorus species is meadow fescue, Schedonorus pratensis.

11. A dihaploid Schedonorus species plant produced by the method of claim 9.

12. (canceled)

13. A method of producing dihaploid festulolium plant material, comprising:
(a) providing a Lolium multiflorum line which is capable of inducing genome loss;
(b) crossing said L. multiflorum line as the maternal parent with a Schedonorus species utilized as the paternal parent to generate F1 interspecific hybrid plants;
(c) identifying sectors in said F1 hybrid plants or in plants which are progeny thereof in which said sectors have a phenotype representative of festulolium comprising a dihaploid festulolium karyotype; and
(d) recovering said sectors identified in (c),
wherein said sectors identified in (c) are capable of developing into mature festulolium plants, and
wherein said Schedonorus species utilized as the paternal parent is not capable of growing rhizomes and at least one of said F1 hybrid plants is capable of growing rhizomes.

14. The method of claim 13, wherein the L. multiflorum line is selected from the group consisting of IL1 (ATCC deposit accession no. PTA-10229) and progeny thereof, and IL2 (ATCC deposit accession no. PTA-10315) and progeny thereof.

15. The method of claim 13, further comprising growing a mature dihaploid festulolium plant from at least one of: one of said F1 hybrid plants or progeny therefrom, or one of said sectors recovered in (d).

16. The method of claim 13, wherein said Schedonorus species is meadow fescue, Schedonorus pratensis.

17. The method of claim 13, wherein at least one of said F1 hybrid plants comprises cytoplasm of L. multiflorum.

18. (canceled)

19. A dihaploid festulolium species plant material produced by the method of claim 13.

20. A method of producing a dihaploid F1 hybrid plant, comprising:
(a) providing a Lolium multiflorum line which is capable of inducing genome loss;
(b) crossing said L. multiflorum line as the maternal parent with a Schedonorus species utilized as the paternal parent to generate interspecific hybrid plants; and
(c) recovering at least one of said F1 hybrid plants that comprises at least one sector having a phenotype representative of Schedonorus comprising a dihaploid Schedonorus karyotype,
wherein said Schedonorus species utilized as the paternal parent is not capable of growing rhizomes and at least one of said F1 hybrid plants is capable of growing rhizomes.

21. The method of producing a dihaploid plant of claim 20, further comprising:
(d) providing the diploid dihaploid F1 plant of step (c);
(e) growing a mature dihaploid Schedonorus plant from at least one of: the F1 hybrid plant, progeny therefrom, or the at least one of said sectors having a phenotype representative of Schedonorus comprising a dihaploid Schedonorus karyotype.

22. The method of claim 20, wherein said Schedonorus species is meadow fescue, Schedonorus pratensis.

23. A dihaploid Schedonorus plant produced by the method of claim 21.

24. (canceled)
